(19) United States
(12) Patent Application Publication
(10) Pub. No.: US 2018/0250649 A1
(43) Pub. Date: Sep. 6, 2018

(54) RADIOPHARMACEUTICAL LABELING DEVICE

(71) Applicants: David ALEXOFF, Westhampton, NY (US); Dohyun KIM, Ridge, NY (US); Hank KUNG, Springfield, PA (US)

(72) Inventors: David ALEXOFF, Westhampton, NY (US); Dohyun KIM, Ridge, NY (US); Hank KUNG, Springfield, PA (US)

(21) Appl. No.: 15/909,000

(22) Filed: Mar. 1, 2018

Related U.S. Application Data
(60) Provisional application No. 62/466,087, filed on Mar. 2, 2017.

(51) Int. Cl. B01J 19/00; G16H 40/40

(52) U.S. Cl. CPC B01J 19/0006; G21G 1/0005; B01J 19/0046; G16H 40/40

(57) ABSTRACT

An automated apparatus and method for producing a 68Ga radiopharmaceutical is provided. The apparatus can direct fluid flow through a radiopharmaceutical generator, a vessel in a temperature controlled reactor, and a solid phase extraction cartridge to produce a final radiopharmaceutical product without human intervention under a clean environment.

### CROSS REFERENCE TO RELATED APPLICATIONS

The present application claims priority to U.S. Provisional App. No. 62/466,087, filed Mar. 2, 2017.

### BACKGROUND

### Field

Embodiments of the present invention relate to devices and methods to manufacture radiopharmaceuticals without human intervention under a clean environment.

### Background

Positron Emission Tomography (PET) is a functional imaging modality that uses a radioactive substance (radiotracer) to reveal the size, shape, position, and some function of organs. Machines are used to manufacture most radiopharmaceuticals used for PET.

The positron-emitting isotope of gallium, Ga-68, can be used as a radiotracer for PET. Ga-68 has a half-life of only 68 minutes. This short half-life renders a radiopharmaceutical using Ga-68 inconvenient for transport. As a result, radiopharmaceuticals using Ga-68 can be manufactured at or near the same location as the PET imaging.

Current devices used to manufacture Ga-68 are often descendants of more complicated systems that support more complex types of chemical operations needed to synthesize a diverse class of radiotracers, often using the cyclotron produced isotopes of Carbon-11 (C-11) and Fluorine-18 (F-18). As such, these systems, when applied to Ga-68 chemistry can be overly complex, less reliable, difficult to implement, and even larger in size than is necessary.

Even systems designed with Ga-68 chemistry in mind are expensive and complicated and continue to follow many of the design characteristics of their complex forefathers used for C-11 and F-18 syntheses. For example, all systems currently available for Ga-68 chemistry require that the user install both the chemistry module (hardware containing the plumbing and some controls) and a controlling device (computer, tablet, etc.) connected usually through a cable (e.g., Ethernet; RS-422; RS-232 serial protocols for example). These interconnect systems between the user and the device require design of penetrations through expensive shielding systems and can be the source of operational problems due to noise pickup on communication cables or lack of synchronization between the communicating devices.

While manually controlled systems can also be used to synthesize Ga-68 radiopharmaceuticals, a human operator must sequence each step in the process. Use of a manual device can expose the operator to radioactivity and introduce error into production.

### BRIEF SUMMARY OF THE INVENTION

Automating the machines and radio-synthesis process to manufacture radiopharmaceuticals can minimize worker exposure to radioactivity; maximize reliability and reproducibility of production by eliminating the need for human execution of repetitive, boring tasks; improve control of sterility by minimizing human contact during processing; and help to standardize methods necessary for commercialization or technology transfer between institutions.

Device and methods described herein mitigate the problems of prior radiopharmaceutical machines. In one aspect of the invention, the device described herein can incorporate an integrated design that permits single pushbutton control, requiring no external device or computer to operate. A user need not learn or use any computer/tablet software to execute the automated process. In addition, secure software controlling automated systems is required by the regulatory agencies overseeing PET radiopharmaceutical manufacturing. In another aspect of the invention, software security can be enhanced because process-controlling information resides only in firmware that cannot be edited or inadvertently changed during normal operation. In addition, the omission of an external controlling device improves reliability by eliminating communication cabling that can be a source of noise pickup.

In one aspect of the invention, an automated method to synthesize a radiopharmaceutical can include passing a fluid through a radioactive labeling generator to introduce the radioactive labeling starting material into a precursor labeling solution, mixing the precursor labeling solution, heating the labeling solution, purifying the solution by solid phase extraction, formulating a final radiopharmaceutical product, and performing sterile filtration of the final radiopharmaceutical product. In the automated method to synthesize a radiopharmaceutical, the radioactive labeling generator can be a 68Ge/68Ga generator, the labeling solution can be heated for approximately 70 degrees Celsius for approximately 5 minutes, and the final radiopharmaceutical product can be formulated by eluting an extracted drug substance using 2 mL of 0.1 N NaOH and mixing with 1 mL of 0.2 N HCl, 6 mL of saline, and approximately 32 mg sodium acetate trihydrate.

In another aspect of the invention, an automated apparatus for producing a radiopharmaceutical can include an outer housing, a temperature controlled reactor, and a valve manifold. The valve manifold can include a plurality of manifold valves and can be detachably attached to the outer housing. The valve manifold can be fluidly connected to a radiopharmaceutical reactor that is fluidly connected to a reactor reservoir containing a generator fluid to elute a material from the radiopharmaceutical reactor, a reaction vessel positioned within the temperature controlled reactor that is configured to heat the reaction vessel, a solid phase extraction device to extract the material from the generator fluid, a reagent reservoir containing a reagent fluid to elute the material from the solid phase extraction device, and a final radiopharmaceutical vessel to receive the reagent fluid and the material.

The automated apparatus can include a high-pressure regulator fluidly connected to a pressure source and a high-pressure valve, a low-pressure regulator fluidly connected to the pressure source and a low-pressure valve, a vacuum pump, and a pressure measurement device. The valve manifold can be fluidly connected to the high-pressure valve, the low-pressure valve, the vacuum pump, and the pressure measurement device. The plurality of manifold valves can be three-way valves. The valve manifold can be a stopcock manifold and each of the plurality of manifold valves can be a multi-way stopcock valve. One of the plurality of manifold valves can include a valve handle and the valve manifold can be detachably attached to the outer housing via a valve adapter that is detachably attached to the valve handle.

The automated apparatus can include a controller to control operation of the high-pressure valve, the low-pressure valve, the vacuum pump, the temperature controlled reactor, and the plurality of manifold valves. During operation of the automated apparatus, the controller can operate without receiving input from a computing device. Operation of the automated apparatus is commenced by an input signal to the controller. The input signal can be generated by a mechanical switch.

The automated apparatus can include a motor attached to the valve adapter to rotate the valve adapter and change a position of the valve handle and an operating position of the valve. The automated apparatus can include a generator fluid pump to move the generator fluid through the radiopharmaceutical reactor and a controller to control operation of the motor and the generator fluid pump such that during operation of the automated apparatus, the controller does not receive input from a computing device.

In another aspect of the invention, a valve adapter to detachably attach a valve handle to a motor can have a length extending along a first direction and a width extending along a second direction, the second direction being perpendicular to the first direction. The valve adapter can include a central body having a valve end and a motor end, the valve end and the motor end being spaced along the first direction. The valve adapter can also include a first cantilever member attached to the central body at the valve end and a first cantilever member first end, the first cantilever member extending from the central body in the second direction to a first cantilever member second end and a second cantilever member attached to the central body at the valve end and a second cantilever member first end, the second cantilever member extending from the central body in the second direction to a second cantilever member second end. The valve adapter can include a protrusion attached to the first cantilever member, the protrusion being positioned between the first cantilever member and the second cantilever member. The protrusion can be configured to engage the valve handle to detachably attach the valve handle to the valve adapter.

The valve adapter can include a second protrusion attached to the second cantilever member. The second protrusion can be positioned between the first cantilever member and the second cantilever member and can be configured to engage the valve handle to detachably attach the valve handle to the valve adapter.

The valve adapter can include a channel to receive a second valve handle, the channel extending along a third direction, the third direction being perpendicular to the first direction and the second direction. The valve adapter can include a dual tapered guide surface positioned adjacent the channel to direct the second valve handle into the channel, the dual tapered guide surface being tapered in the first direction and in the third direction. The valve adapter can include a second channel to receive a third valve handle, the second channel extending along the third direction.

The valve adapter can include a spline inset in the motor end of the central body that is configured to engage a gear of the motor.

Further features and advantages of embodiments of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to a person skilled in the relevant art(s) based on the teachings contained herein.

### BRIEF DESCRIPTION OF THE DRAWINGS/FIGURES

The accompanying drawings, which are incorporated herein and form part of the specification, illustrate embodiments of the present invention and, together with the description, further serve to explain the principles of the invention and to enable a person skilled in the relevant art(s) to make and use the invention.

FIG. 1 is a front perspective view of a radiopharmaceutical labeling device according to various aspects of the invention.

FIG. 2 is a top view of a radiopharmaceutical labeling device according to various aspects of the invention.

FIG. 3 is a rear perspective view of a radiopharmaceutical labeling device according to various aspects of the invention.

FIG. 4 is a front view of a radiopharmaceutical labeling device according to various aspects of the invention.

FIG. 5 is a flow diagram of a radiopharmaceutical labeling device according to various aspects of the invention.

FIG. 6 is a perspective view of a manifold adapter according to various aspects of the invention.

FIG. 7 is a top view of a manifold adapter according to various aspects of the invention.

FIG. 8 is a bottom view of a manifold adapter according to various aspects of the invention.

FIG. 9 is a side view of a manifold adapter according to various aspects of the invention.

FIG. 10 is a front perspective view of manifold adapters, motors, and a mounting plate according to various aspects of the invention.

FIG. 11 is a front perspective view of manifold adapters, cassettes, motors, and a mounting plate according to various aspects of the invention.

FIG. 12 is a perspective view of a generator source reservoir holder according to various aspects of the invention.

FIG. 13 illustrates an example hardware platform according to various aspects of the invention.

FIG. 14 is a block diagram of an example method for operating a radiopharmaceutical labeling device according to various aspects of the invention.

FIG. 15 is a block diagram of an example method for operating a radiopharmaceutical labeling device according to various aspects of the invention.

FIG. 16 is a block diagram of an example method for operating a radiopharmaceutical labeling device according to various aspects of the invention.

FIG. 17 illustrates an example hardware platform according to various aspects of the invention.

Features and advantages of the embodiments will become more apparent from the detailed description set forth below when taken in conjunction with the drawings, in which like reference characters identify corresponding elements throughout.

### DETAILED DESCRIPTION OF THE INVENTION

The present invention(s) will now be described in detail with reference to embodiments thereof as illustrated in the accompanying drawings. References to “one embodiment”, “an embodiment”, “an exemplary embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In one aspect of the invention, the present device and methods described herein can include a number of features and benefits to provide formulation of radiopharmaceutical suitable for intravenous injection. For example, the device can have a compact size to minimize use of expensive shielding. The device can operate autonomously after initiation on a simplified user input. A syringe pump can be utilized for elution of a radiopharmaceutical generator. The device can also be configurable for generator fractionation. In a further aspect, the labeling solution can be mixed by gas bubbling. The labeling solution can also be heated using a temperature controller. In another aspect, the device can utilize solid phase purification that can be configured for purification of the radio pharmaceutical before or after labeling. The radio pharmaceutical final product can be sterilized by filtration.

The simplified radiolabeling procedures associated with Ga-68 radiopharmaceuticals, afford an opportunity to optimize machine design, tailoring the capabilities of an automated device to the specific needs of Ga-68 chemistry. For example, in Ga-68 applications the radioactive starting material is introduced through elution of a Ge-68/Ga-68 generator and therefore a Ga-68 machine can incorporate this functionality. To accommodate the broadest range of Ga-68 applications, the system can be configurable to include fractionation during elution of the generator, and the purification can be configurable for either purification of the generator elution solution before labeling or purification of the labeling solution after labeling.

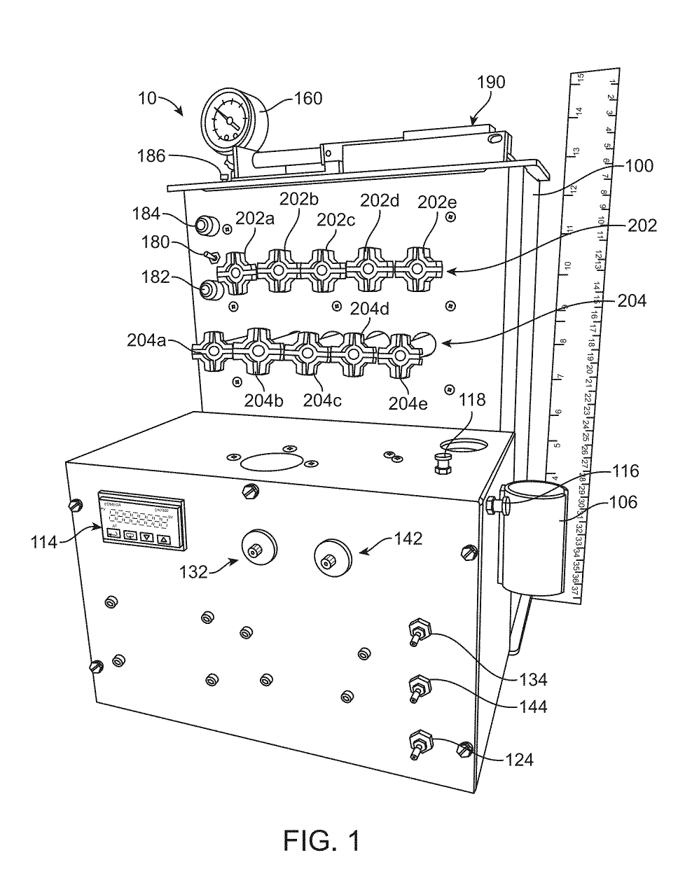

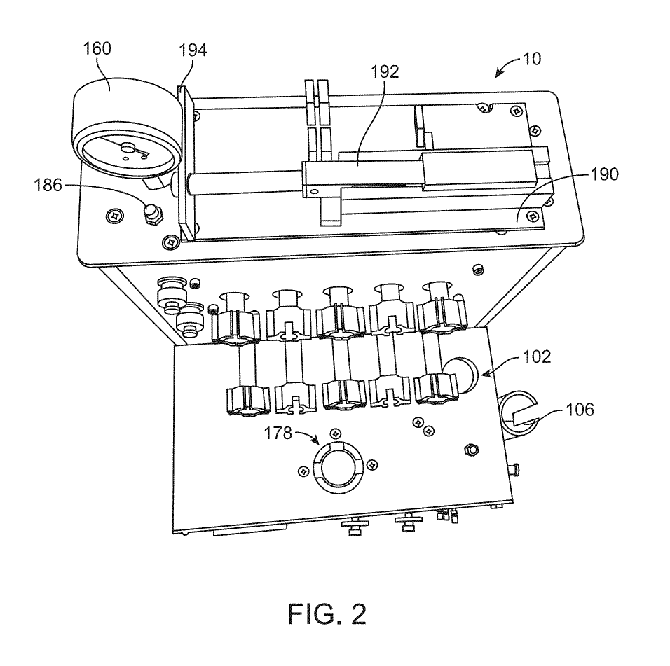

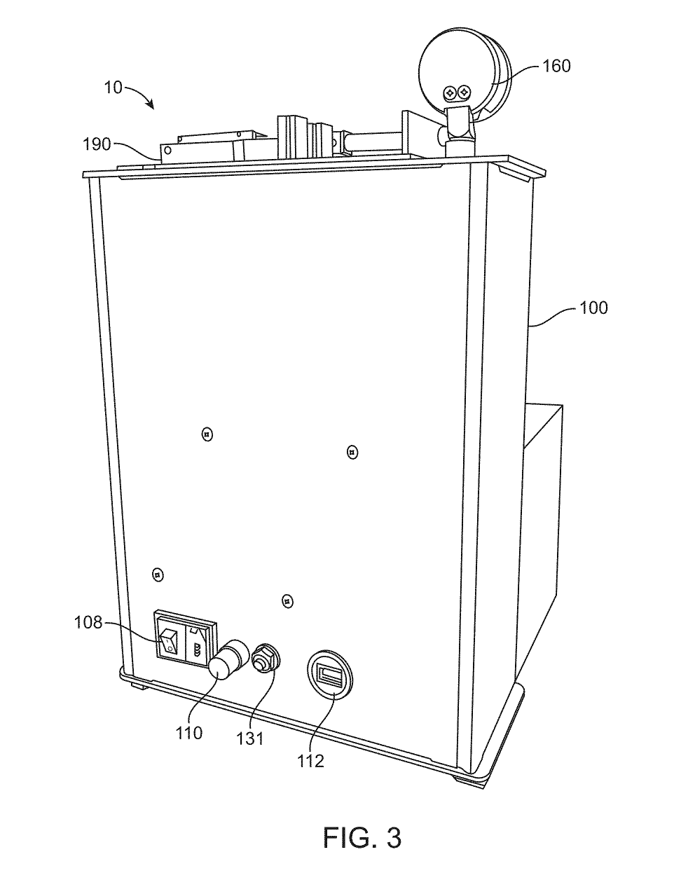

Radiopharmaceutical labeling device 10 is shown in FIGS. 1-3. As discussed in further detail below, device 10 can include housing 100, temperature controlled reactor 102 to heat a reaction vessel, a fan 104 to cool a reaction vessel, a reactor vacuum trap holder 106, a power switch and cable connector 108, a fuse holder 110, and a data transmission port 112. Device 10 can also include tubing connector 131, temperature controller 112, waste pressure control port 116, reactor pressure control port 118, vacuum needle valve 124, high-pressure needle valve 134, and low-pressure needle valve 144. Device 10 can include high-pressure regulator 132, low-pressure regulator 142, pressure gauge 160, buffer vial holder 178, generator source reservoir holder 190, linear actuator 192, push plate 194, and syringe barrel restraint members 196. Device 10 can include vacuum pump 120, vacuum pump muffler 122, vacuum two-way solenoid 126, high-pressure two-way solenoid 136, low-pressure two-way solenoid 146, vent two-way solenoid 156, waste vessel three-way solenoid valve 166, and reaction vessel three-way solenoid valve 176. Device 10 can further include upper manifold stopcock adapters 202 including upper manifold stopcock adapters 202a-e and lower manifold stopcock adapters 204 including lower manifold stopcock adapters 204a-e. Device can include an upper cassette 301 having manifold valves 302a-302e and a lower cassette 303 having manifold valves 304a-304e. The manifold valves 302a-302e and/or 304a-304e can be three-way or four-way valves. Device 10 can include selector switch 180, start switch 186, test indicator 182, and run indicator 184. Device 10 can include a radio pharmaceutical generator 300, reaction vessel 310, waste vessel 312, buffer vessel 314, and final product vessel 316. Device 10 can further include a solid phase cartridge 320, a first reagent reservoir 330, a second reagent reservoir 332, a third reagent reservoir 334, a vacuum trap 336, and a generator source reservoir 340.

Device 10 can include fluid conduits (e.g., tubing), valves, and components that are not exposed to radioactive material and do not require cleaning and/or disposal with each radiopharmaceutical generation. These fluid conduits and valves can be positioned within an interior area of housing 100, on housing 100, or attached and/or fluidly to another component of device 10. For example, vacuum pump 120, vacuum needle valve 124, vacuum two-way solenoid valve 126, pressure source 130, high-pressure regulator 132, high-pressure needle valve 134, high-pressure two-way solenoid valve 136, low-pressure regulator 142, low-pressure needle valve 144, low-pressure two-way solenoid valve 146, vent two-way solenoid valve 156, pressure gauge 160, waste vessel three-way solenoid valve 166, reaction vessel three-way solenoid valve 176, and the fluid conduits connecting these respective components are not exposed to radioactive material and do not require cleaning and/or disposal with generation of each radiopharmaceutical product.

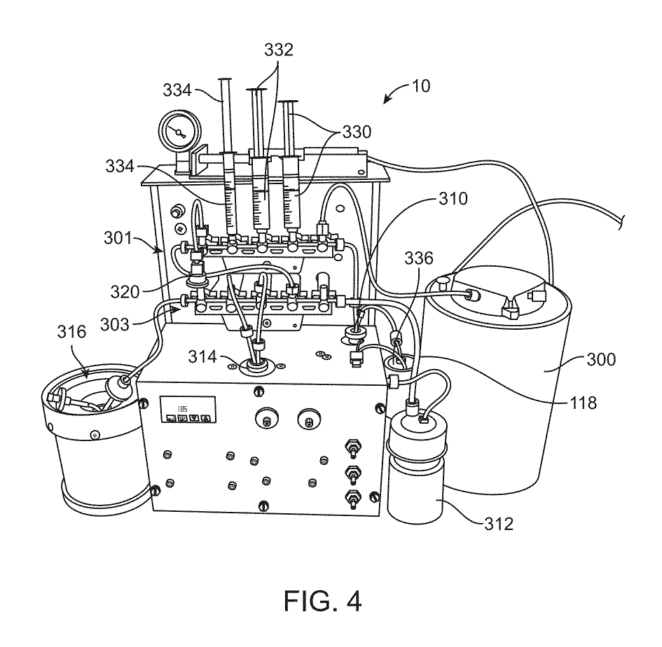

Device 10 can also include fluid conduits and valves that are exposed to radioactive material during radiopharmaceutical generation. All components that contact drug product materials can be single-use and disposable. In one aspect, device 10 can operate on cassette-based system that enhances sterility control and eliminates the need for validated cleaning procedures. For example, device 10 can utilize two disposable 5-ganged stopcock manifolds for upper cassette 301 and lower cassette 303 (FIG. 4). Such a cassette-based system permits the use of different radionuclides in the same device by eliminating the potential for cross-contamination. For example, the disclosed device can prepare different Ga-68 radiopharmaceuticals without a change in firmware. Upper cassette 301 and lower cassette 303 and the fluid conduits connected to these cassettes can be sterilized and/or disposed of after generation of a radiopharmaceutical product. In one aspect, upper cassette 301, lower cassette 303, and fluid conduits 402, 404, 406, 408, 410, 412, 414, 416, 418, 420, 422, and 424 can be disposable. Manifold valves 302a-302e and/or 304a-304e can be three-way valves. In another aspect, Upper cassette 301 and/or lower cassette 303 can be a stopcock manifold and each manifold valves 302a-302e and/or 304a-304e can be multi-way stopcock valves.

Device 10 can be utilized with a hardware kit. The kit can include upper cassette 301, lower cassette 303, reaction vessel 310 that can have lyophilized reagents, waste vessel 312, buffer vessel 314, final product vessel 316, solid phase cartridge 320, first reagent reservoir 330, second reagent reservoir 332, third reagent reservoir 334, vacuum trap 336, generator source reservoir 340, fluid conduit 402, fluid conduit 404, fluid conduit 406, fluid conduit 408, fluid conduit 410, fluid conduit 412, fluid conduit 414, fluid conduit 416, fluid conduit 418, fluid conduit 420, fluid conduit 422, and fluid conduit 424.

Selector switch 180 can be positioned on housing 100. Selector switch 180 can be a multi-position switch, for example a toggle switch. Selector switch 180 can have a first position representative of a “test mode” and a second position representative of a “run mode” to generate a radiopharmaceutical. The test and run modes are described in greater detail below.

Test indicator 182 and run indicator 184 can also be positioned on housing 100. Test indicator 182 can be correlated with the first position of selector switch 180 to alert the user that device 10 is in the test mode. Test indicator 182 can be a visual indicator, for example a light source. In one aspect, test indicator 182 can be a colored light emitting diode (LED), for example, a yellow LED. Run indicator 184 can be correlated with the second position of selector switch 180 to alert the user that device 10 is in the run mode. Run indicator 184 can be a visual indicator, for example a light source. In one aspect, run indicator 184 can be a colored LED, for example, a green LED.

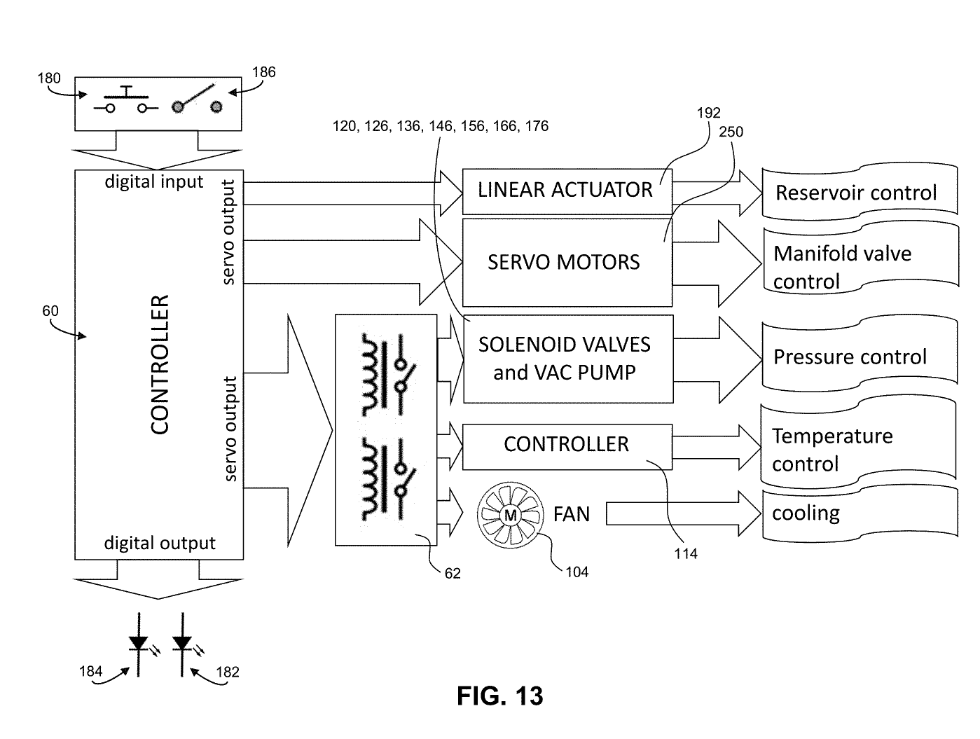

Start switch 186 can be positioned on housing 100 to initiate operation of device 10. Start switch 186 can be a mechanical switch such as a pushbutton switch or a rocker switch. Selector switch 180 and start switch 186 can be connected to a controller (FIG. 13) to control operation of device 10. For example, selector switch 180 and start switch 186 can initiate operation of device 10 in the test mode or the run mode. In one aspect of the invention, device 10 is not connected to an external computing device to control operation of device 10 in the test mode or the run mode.

Device 10 can include data transmission connector 112 to interface device 10 with a computer for firmware updates to the controller. For example, the controller firmware of device 10 can be updated via data transmission connector 112 to modify the sequence for the test mode or the run mode. In another aspect, the controller firmware can be updated via data transmission controller 112 to modify the operation of device 10 based on a desired radiopharmaceutical.

Temperature controlled reactor 102 can be positioned in housing 100, for example, within an interior area of housing 100. In another aspect, temperature controlled reactor 102 can be attached to an exterior surface of housing 100. Device 10 can also include a temperature controller 114 to operate temperature controlled reactor 102 to heat reaction vessel 310 to a specified temperature. In one aspect, temperature controlled reactor 102 can have a heating capability greater than approximately 120 degrees Celsius. Temperature controller 114 can maintain a desired temperature within temperature controlled reactor 102 within a range of approximately three degrees Celsius above and/or below the desired temperature, for example, using its on/off control mode. A fan (FIG. 13) can be positioned adjacent temperature controlled reactor 102 to cool reaction vessel 310 after heating in temperature controlled reactor 102.

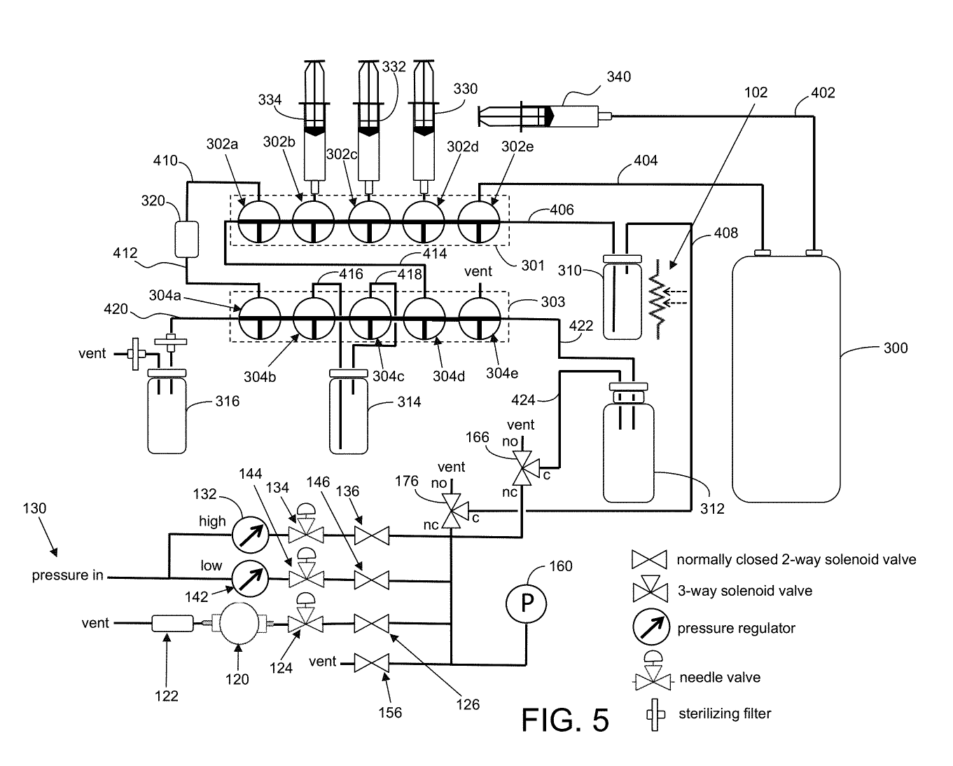

Device 10 can use vacuum and/or pressure for fluid transfer within the components of device 10. In one aspect, pressure source 130 (FIG. 5) can be utilized for fluid transfer. Pressure source 130 can be nitrogen or other sterile gas. Pressure source 130 can be at a pressure of approximately 60 psig to approximately 100 psig. Pressure source 130 can be fluidly connected to high-pressure regulator 132 and low pressure regulator 142 via one or more fluid conduits. Pressure regulators 132 and 142 can be set during calibration of device 10. In one aspect, high-pressure regulator can be at approximately 13 psig to approximately 16 psig. Low-pressure regulator can be at approximately 8 psig to approximately 11 psig.

As shown in FIGS. 4-5, high-pressure regulator 132 can be fluidly connected to high-pressure needle valve 134 and a normally closed high-pressure two-way solenoid valve 136. Low-pressure regulator 143 can be fluidly connected to low-pressure needle valve 144 and a normally closed low-pressure two-way solenoid valve 146.

Device 10 can include a vacuum pump 120 for fluid transfer control. Vacuum pump 120 can provide vacuum up to approximately 26 mmHg. Vacuum pump muffler 122 can reduce the noise associated with operating vacuum pump 120. Vacuum pump 120 can be fluidly connected to vacuum needle valve 124 and a normally closed vacuum two-way solenoid valve 126.

A vent can be fluidly connected to vent two-way solenoid valve 156 to dissipate and/or equilibrate the pressure within the system of device 10.

Waste vessel three-way solenoid valve 166 can be fluidly connected to vacuum two-way solenoid valve 126, high-pressure two-way solenoid valve 136, low-pressure two-way solenoid valve 146, and vent two-way solenoid valve 156. Valve 166 can be fluidly connected to a vent to dissipate pressure in the system of device 10. Valve 166 can also be fluidly connected to waste vessel 312 to move fluid through the system of device 10. In another aspect, valve 166 can be fluidly connected to reaction vessel three-way solenoid valve 176. In a further aspect of the invention, valve 166 can be fluidly connected to waste pressure control port 116 on housing 10. Waste pressure control port 116 can be fluidly connected to waste vessel 312, for example, using a disposable conduit or tubing.

Reaction vessel three-way solenoid valve 176 can be fluidly connected to vacuum two-way solenoid valve 126, high-pressure two-way solenoid valve 136, low-pressure two-way solenoid valve 146, and vent two-way solenoid valve 156. Valve 176 can be fluidly connected to a vent to dissipate pressure in the system of device 10. Valve 176 can also be fluidly connected to reaction vessel 310 to move fluid through the system of device 10. In a further aspect of the invention, valve 176 can be fluidly connected to reactor pressure control port 118 on housing 10. Reactor pressure control port 118 can be fluidly connected to reaction vessel 310, for example, using a disposable conduit or tubing.

As shown in FIG. 5, generator source reservoir 340 can be fluidly connected to radiopharmaceutical generator 300 through fluid conduit 402. Radiopharmaceutical generator 300 can be fluidly connected to manifold valve 302e through fluid conduit 404. Manifold valve 302e can be fluidly connected to reaction vessel 310 through fluid conduit 406. Reaction vessel 310 can be fluidly connected to reaction vessel three-way solenoid valve 176 through fluid conduit 408. First reagent reservoir 330 can be fluidly connected to manifold valve 302d. Second reagent reservoir 332 can be fluidly connected to manifold valve 302c. Third reagent reservoir 334 can be fluidly connected to manifold valve 302b. Manifold valve 302a can be fluidly connected to manifold valve 304d through fluid conduit 414. Manifold valve 302a can be fluidly connected to a solid phase cartridge 320 through fluid conduit 410. Solid phase cartridge 320 can be fluidly connected to manifold valve 304a through fluid conduit 412. Manifold valve 304a can be fluidly connected to a final product vessel 316 through fluid conduit 420. Manifold valve 304b can be fluidly connected to buffer vessel 314 through fluid conduit 416. Buffer vessel 314 can be fluidly connected to manifold valve 304c through fluid conduit 418. Manifold valve 304e can be fluidly connected to waste vessel 312 through fluid conduit 422. Waste vessel 312 can be fluidly connected to waste vessel three-way solenoid valve 166 through fluid conduit 424.

Pressure gauge 160 can be fluidly connected to one or more of vacuum two-way solenoid valve 126, high-pressure two-way solenoid valve 136, low-pressure two-way solenoid valve 146, vent two-way solenoid valve 156, waste vessel three-way solenoid valve 166, and reaction vessel three-way solenoid valve 176. Pressure gauge 160 can be used in the test mode and the run mode to confirm the desired operating pressures within the components of device 10, for example, in the fluid conduits and/or upper cassette 301 and lower cassette 303. Pressure gauge can measure ±30 mmHg. Pressure gauge 160 can be attached to device housing. Pressure gauge 160 can be a Bourdon type gauge having a needle and dial readout.

Device 10 can also include one or more manifold adapters 200. Manifold adapters can be upper manifold adapters 202 and/or lower manifold adapters 204. As shown in FIG. 1, device 10 can include five upper manifold adapters 202a-202e and five lower manifold adapters 204a-204e. Any number of upper manifold adapters 202 and/or lower manifold adapters 204 can be utilized based on the desired number of manifold valves for appropriate fluid flow through device 10. When used together, one of upper manifold adapters 202 and lower manifold adapters 204 can have an elongated shaft 232 for ease of operation and connection of components to upper cassette 301 and lower cassette 303.

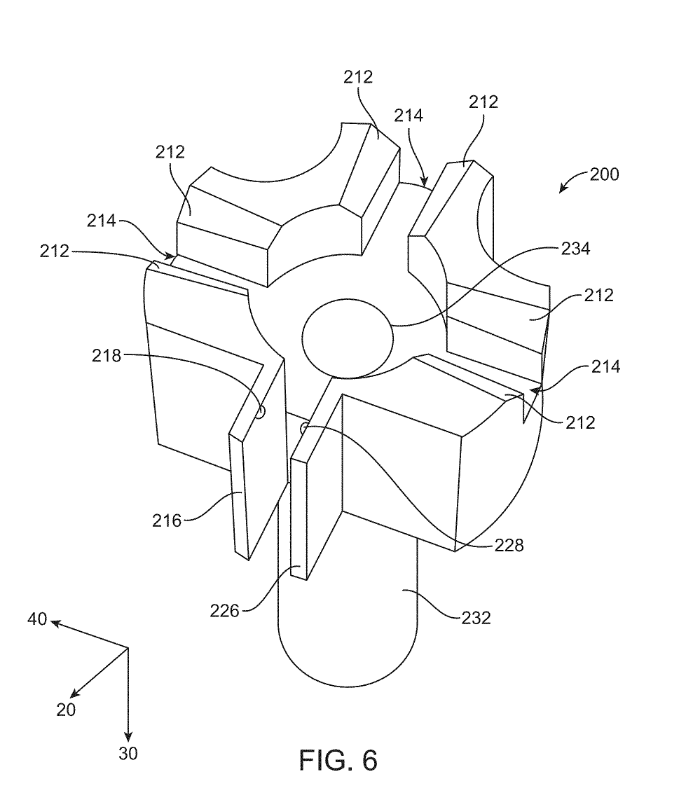

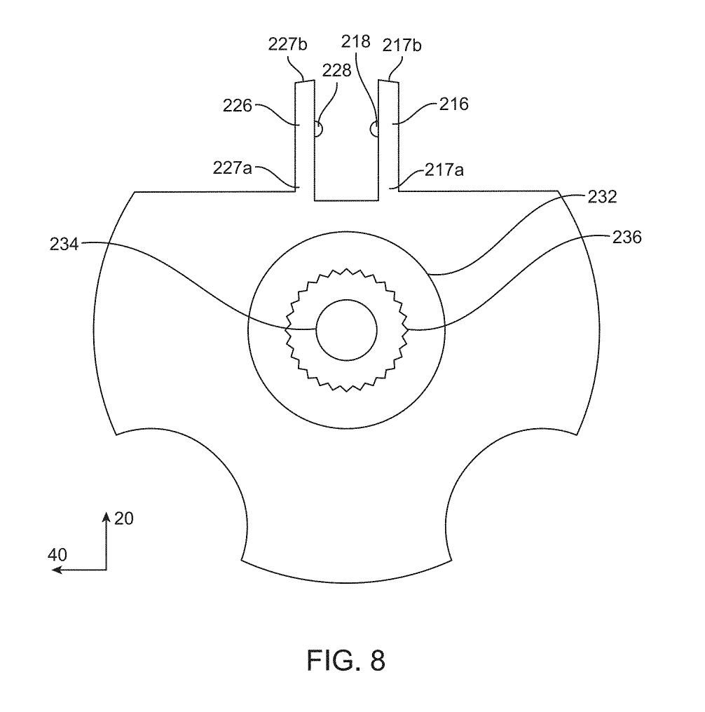

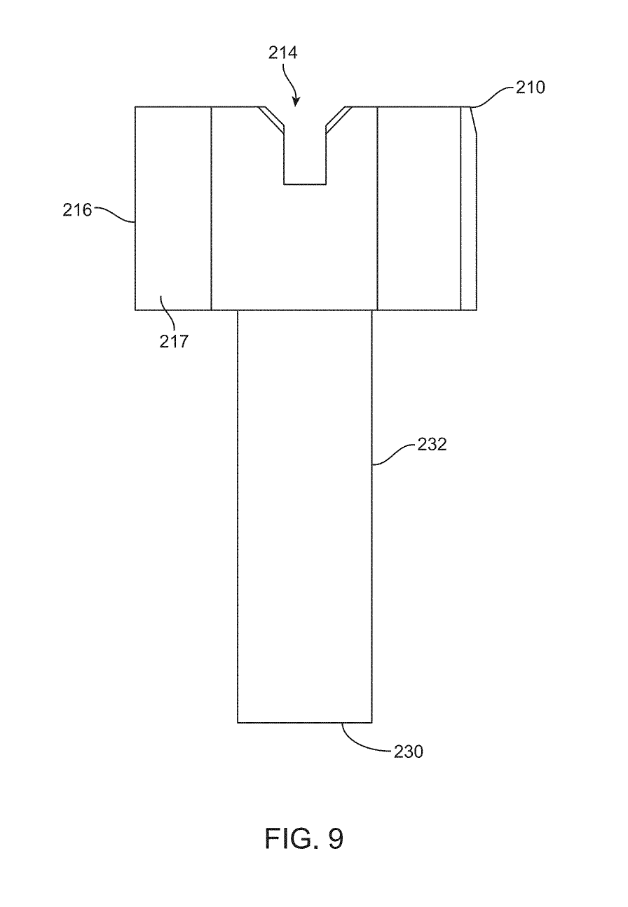

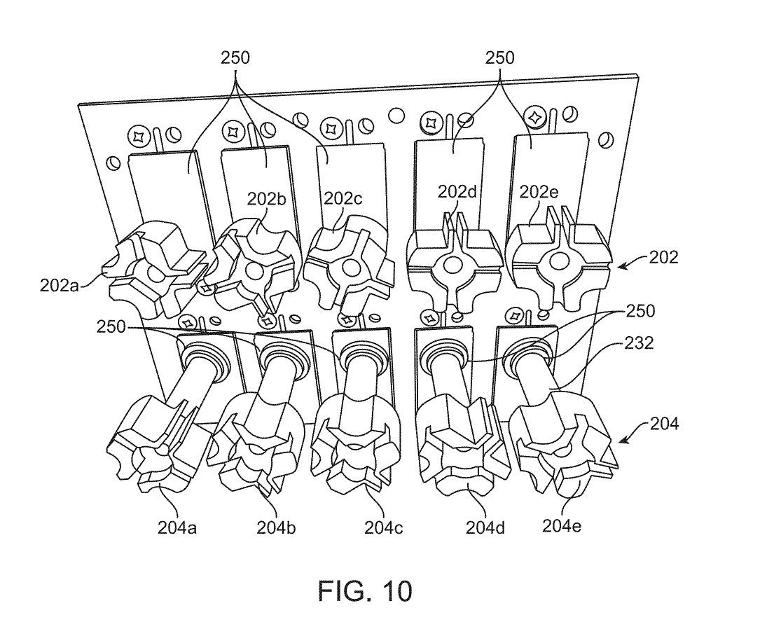

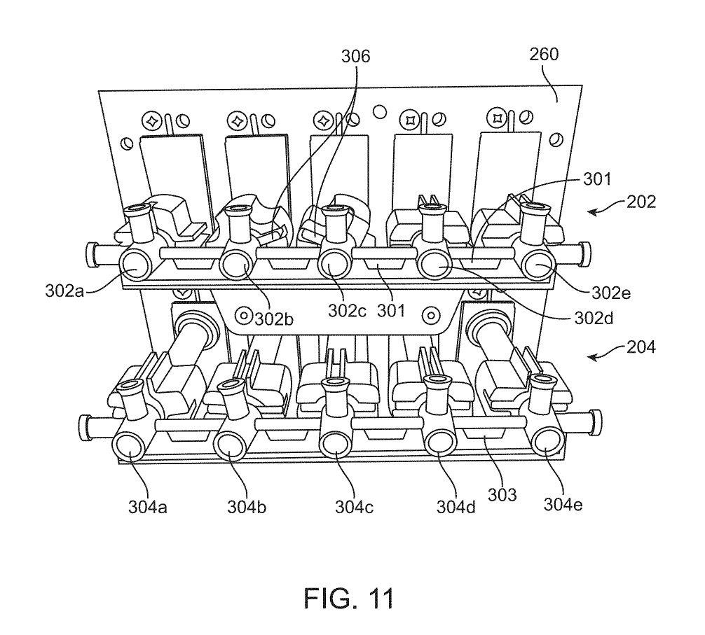

Each manifold adapter 200 that makes up the upper manifold adapters 202 and the lower manifold adapters 204 can include a valve end 210 and a motor end 230 separated by a shaft 232 (FIGS. 6-9). Shaft 232 can be cylindrical. A spline 236 can be positioned within shaft 232 at motor end 230 (FIG. 8). Spline 236 can directly interface with a motor gear attached to a motor 250. Spline 236 can limit the need for additional mechanical couplings or other components to attach manifold adapter 200 to motor 250. Motor 250 can be attached to a mounting plate 260 for attachment to device 10 (FIGS. 10-11).

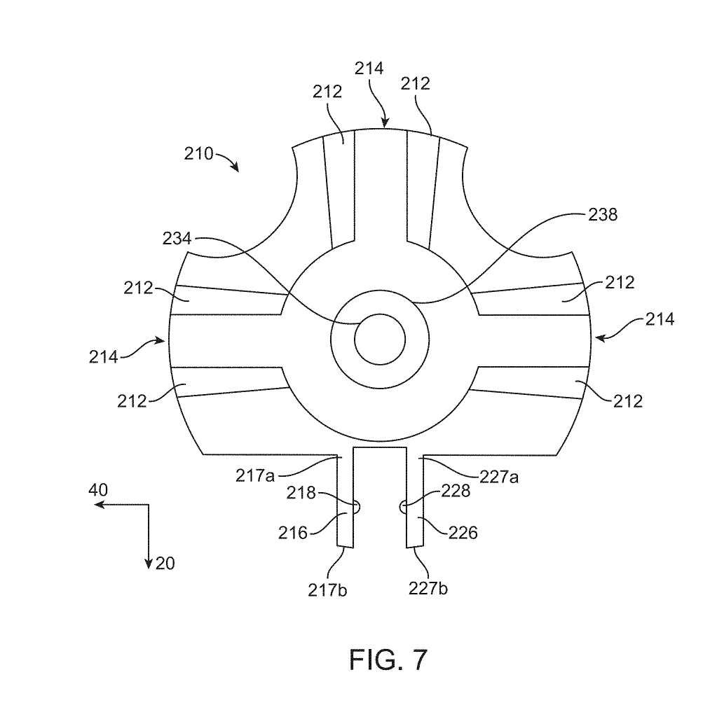

As shown in FIGS. 6-7, manifold adapter 200 also includes a through hole 234 and a ledge 238 so manifold adapter can be fixed to the motor 250 using a screw, bolt, or other attachment means.

Manifold adapter 200 can include one or more channels 214. In one aspect, manifold adapter 200 can include three channels 214. Channels 214 can receive a manifold valve handle 306 such that manifold valve handle 306 is positioned within a respective channel 214. Manifold adapter 200 can include dual tapered guide surfaces 212 to direct a manifold valve handle 306 into channel 214. Dual tapered guide surfaces 212 can be tapered along direction 30 and along direction 40 to direct a manifold valve handle 306 into channel 214.

Manifold adapter 200 can be positioned in an “engagement” position and can be configured to engage a manifold valve handle 306 and to turn the valve handle to change the operation position of the manifold valve. For example, manifold adapter 200 can include a cantilever member 216 that extends radially outward from shaft 232 along direction 20. Cantilever member 216 can have a first end 217a adjacent shaft 232 and can extend outward along direction 20 to a second end 217b. A protrusion 218 can be positioned on cantilever member 216. Protrusion 218 can engage a manifold valve handle 306 to detachable attach a manifold valve 302 to the manifold adapter 200. In one aspect of the invention, a manifold valve 302 can be pushed toward manifold adapter 200 so a manifold valve handle 306 engages protrusion 218 and causes cantilever member 216 to elastically deform. Once the manifold valve handle 306 passes beyond protrusion 218, cantilever member 216 can return to its original position. Protrusion 218 can then retain the manifold valve handle 306 as detachably attached to manifold adapter 200. The manifold valve 302 can be removed from manifold adapter 200 by pulling the manifold valve 302 away from manifold valve adapter 200. Manifold valve handle 306 can engage protrusion 218 to cause cantilever member 216 to elastically deform for adequate removal of the manifold valve 302.

In another aspect of the invention, manifold adapter 200 can include a second cantilever member 226 that extends radially outward from shaft 232 along direction 20. Cantilever member 226 can have a first end 227a adjacent shaft 232 and can extend outward along direction 20 to a second end 227b. A protrusion 228 can be positioned on cantilever member 226. Protrusion 228 can engage a manifold valve handle 306 along with cantilever member 216 and protrusion 218 to detachable attach a manifold valve 302 to the manifold adapter 200. In one aspect of the invention, a manifold valve 302 can be pushed toward manifold adapter 200 so a manifold valve handle 306 engages protrusion 218 and protrusion 228 and causes cantilever member 216 and cantilever member 226 to elastically deform. Once the manifold valve handle 306 passes beyond protrusion 218 and protrusion 228, cantilever member 216 and second cantilever member 228 can return to their original position. Protrusions 218 and 228 can then retain the manifold valve handle 306 as detachably attached to manifold adapter 200. The manifold valve 302 can be removed from manifold adapter 200 by pulling the manifold valve 302 away from manifold valve adapter 200. Manifold valve handle 306 can engage protrusions 218 and 228 to cause cantilever members 216 and 226 to elastically deform for adequate removal of the manifold valve 302.

The use of cantilever member 216 and protrusion 218 and/or cantilever member 226 and protrusion 228 can eliminate the need for external clamping to attach manifold valves to manifold adapters 200 and provide tactile feedback to a user during installation. Cantilever member 216 and protrusion 218 and/or cantilever member 226 and protrusion 228 can also permit fast and secure installation of upper cassette 301 and/or lower cassette 303 onto device 10.

If manifold valve 302 is part of a cassette based valve system, for example, upper cassette 301 and/or lower cassette 303, the cassette can be detachably attached to one or more manifold adapters 200 by engaging one or more manifold valve handles 306 with the cantilever members and protrusions of one or more manifold adapters 200 (FIGS. 10-11). In one aspect shown in FIG. 11, manifold valve handles 306 on manifold valves 302a-302e can be detachably attached to upper manifold adapters 202a-202e, respectively. Manifold valve handles 306 on manifold valves 304a-304e can be detachably attached to lower manifold adapters 204a-204e, respectively.

In another aspect, one or more of manifold adapters 202a-202e and/or 204a-204e can be positioned in a “neutral” position such that the respective manifold valve handles 306 of the respective manifold valves 302a-302e and/or manifold valves 304a-304e do not to engage the cantilever members and protrusions on the respective manifold adapters. Rather, the respective manifold valve handles 306 will instead be positioned within channel 214. For example, the manifold valve handle 306 of upper manifold valve 302a can be positioned to engage cantilever member 216 and protrusion 218 and/or second cantilever member 226 and protrusion 228 of upper manifold valve adapter 202a. Upper manifold valve adapters 202b-202e can be positioned in a neutral position such that the manifold valve handles 306 of upper manifold valves 302b-302e do not to engage the respective cantilever members and protrusions of upper manifold valve adapters 202b-202e. Modifying the number of valve handles that engage the respective cantilever members and protrusions of the manifold adapters can modify the force required to detachably attach and detach the cassettes to/from the manifold adapters and device 10. As such, the tactile feedback provided to the user can be modified based on the number of manifold adapters 202a-202e and/or 204a-204e that are positioned in a neutral position. Use of manifold adapters 202a-202e and/or 204a-204e in an engagement position or a neutral position can also provide spare parts in the case of material fatigue and/or failure as the manifold adapter components wear over time.

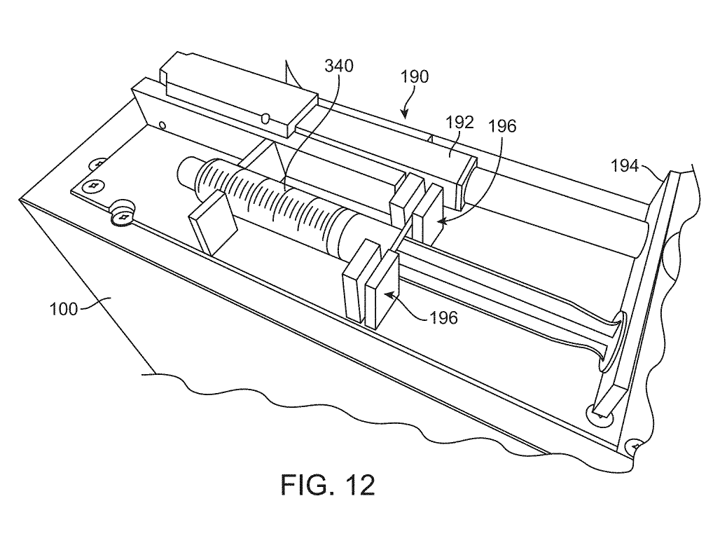

As shown in FIG. 12, generator source reservoir holder 190 can be attached to device housing 100. Generator source reservoir holder 190 can include linear actuator 192 attached to a push plate 194. Push plate 194 can exert a force on generator source reservoir 340 to expel a fluid from generator source reservoir 340 into radiopharmaceutical generator 300. In one aspect, generator source reservoir 340 can be a syringe and push plate 194 can exert a force on a plunger of the syringe. The syringe barrel can be retained on generator source reservoir holder 190 with syringe barrel restraint members 196 positioned on either side of the syringe barrel.

A block diagram showing control of the components in device 10 is shown in FIG. 13. Device 10 can be operated using a controller 60 to provide all necessary functionality to interface with a user and operate autonomously. No cable or wireless connection to an external computing device is required for operation of device 10. As shown, selector switch 180 and start switch 186 can provide an input signal to controller 60. Controller 60 can be incorporated into device 10. Controller can be configured to cause components within device 10 to operate in a desired manner. Controller 60 can provide output signals to test indicator 182 and to run indicator 184 to control operation of test indicator 182 and run indicator 184. Controller 60 can provide an output signal to linear actuator 192 to control the position of linear actuator 192 and operation of generator source reservoir 340. Controller 60 can provide output signals to servo motors 250 to control the position of servo motors 250 and operation of manifold valves 302a-302e and/or manifold valves 304a-304e. Controller 60 can provide output signals to relay board 62 to control operation of vacuum pump 120, vacuum two-way solenoid valve 126, high-pressure two-way solenoid valve 136, low-pressure two-way solenoid valve 146, vent two-way solenoid valve 156, wasted vessel three-way solenoid valve 166, reaction vessel three-way solenoid valve 176, temperature controller 114, and fan 104.

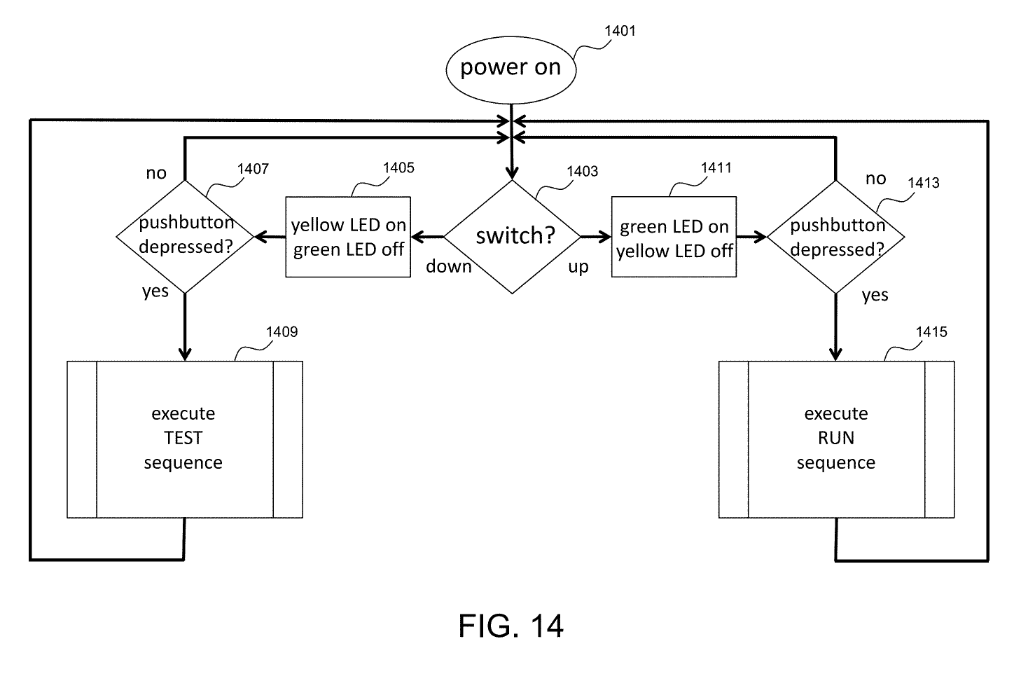

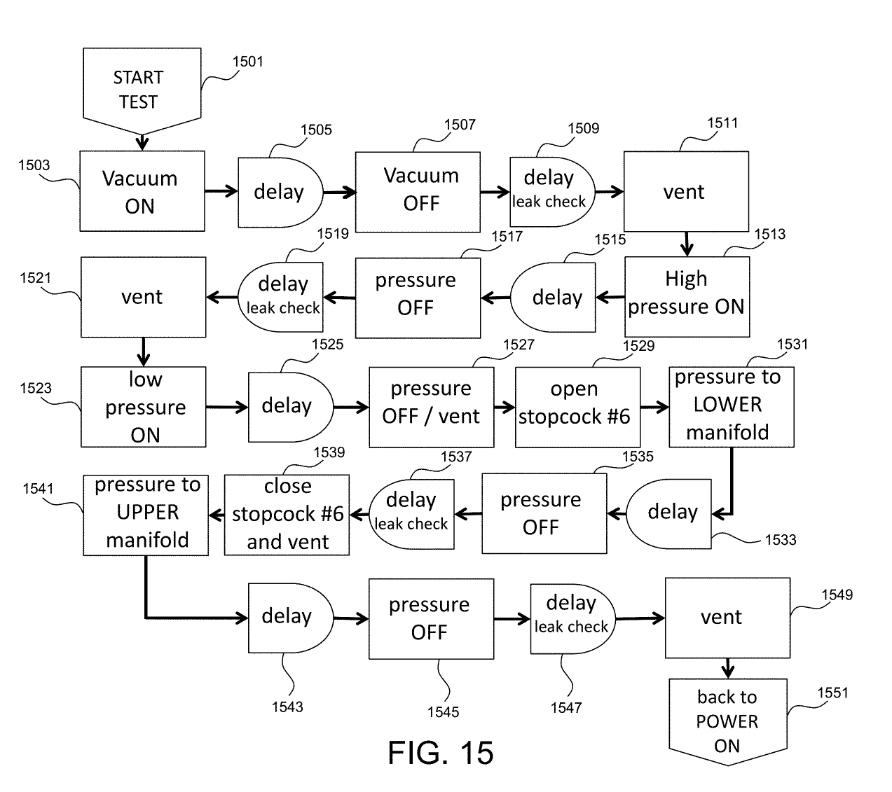

Controller 60 can execute a firmware resident program to control the sequencing and timing of the test and radiopharmaceutical steps discussed with respect to FIGS. 14-15, below.

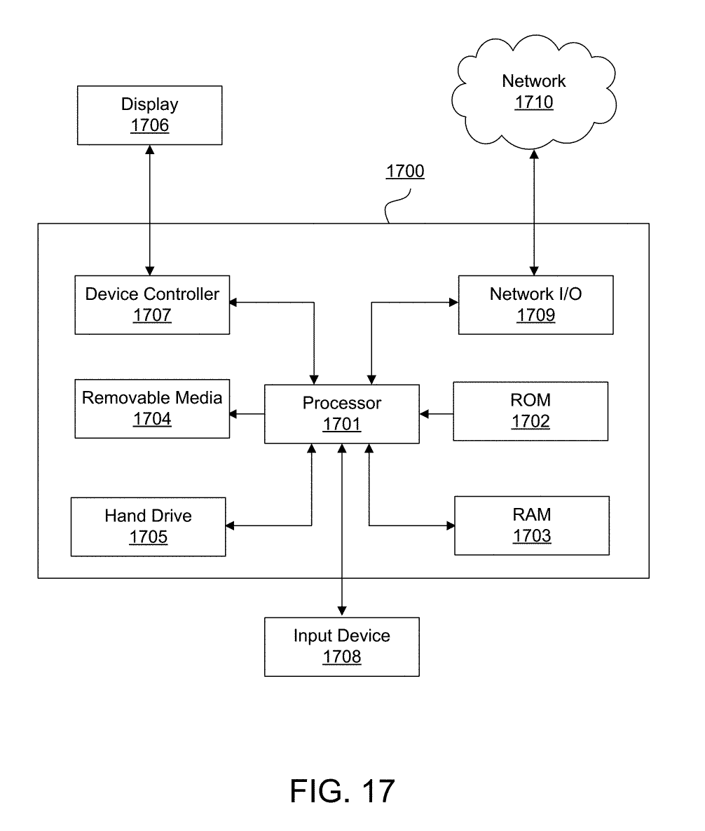

FIG. 17 illustrates an example computing device on which at least some of the various elements described herein can be implemented, including, but not limited to, various components of labeling device 10. Computing device 1700 may include one or more processors 1701, which may execute instructions of a computer program to perform, or cause to perform, any of the steps or functions described herein. The instructions may be stored in any type of computer-readable medium or memory, to configure the operation of the processor 1701. For example, instructions may be stored in a read-only memory (ROM) 1702, random access memory (RAM) 1703, removable media 1704, such as a Universal Serial Bus (USB) drive, compact disk (CD) or digital versatile disk (DVD), floppy disk drive, flash card, or any other desired electronic storage medium. Instructions may also be stored in an attached (or internal) hard drive 1705.

Computing device 1700 may include one or more output devices, such as a display 1706, and may include one or more output device controllers 1707, such as controller 60 and/or a video processor. There may also be one or more user input devices 1708, such as selector switch 180, start switch 186, a touch screen, remote control, keyboard, mouse, microphone, card reader, RFID reader, etc. The computing device 1700 may also include one or more network interfaces, such as input/output circuits 1709 to communicate with an external network 1710. The network interface may be a wired interface, wireless interface, or a combination of the two. In some embodiments, the interface 1709 may include a modem (e.g., a cable modem), and network 1710 may include a desired network.

The FIG. 17 example is an illustrative hardware configuration. Modifications may be made to add, remove, combine, divide, etc. components as desired. Additionally, the components illustrated may be implemented using basic computing devices and components, and the same components (e.g., processor 1701, storage 1702, user input device 1708, etc.) may be used to implement any of the other computing devices and components described herein.

One or more aspects of the disclosure may be embodied in a computer-usable data and/or computer-executable instructions, such as in one or more program modules, executed by one or more computers or other devices. Generally, program modules include routines, programs, objects, components, data structures, etc. that perform particular tasks or implement particular abstract data types when executed by a processor in a computer or other data processing device. The computer executable instructions may be stored on one or more computer readable media such as a hard disk, optical disk, removable storage media, solid state memory, RAM, etc. The functionality of the program modules may be combined or distributed as desired in various embodiments. In addition, the functionality may be embodied in whole or in part in firmware or hardware equivalents such as integrated circuits, field programmable gate arrays (FPGA), controllers, application-specific integrated circuits (ASICS), combinations of hardware/firmware/software, and the like. Particular data structures may be used to more effectively implement one or more aspects of the invention, and such data structures are contemplated within the scope of computer executable instructions and computer-usable data described herein.

Prior to operation of device 10, the components of device 10 can be installed, as shown for example, in FIGS. 4-5. Upper manifold 301 including manifold valves 302a-302e can be installed onto device 10 by detachably attaching one or more of the manifold valve handles 306 to one or more of upper manifold adapters 202a-202e. Lower manifold 303 including manifold valves 304a-304e can be installed onto device 10 by detachably attaching one or more of the manifold valve handles 306 to one or more lower manifold adapters 204a-204e. Generator source reservoir 340 can be fluidly connected to fluid conduit 402, first reagent reservoir 330 can be fluidly connected to manifold valve 302d, second reagent reservoir 332 can be fluidly connected to manifold valve 302c, and third reagent reservoir 334 can be fluidly connected to manifold valve 302b. Fluid conduit 402 can be connected to generator source reservoir 340 and generator 300. Fluid conduit 404 can be connected to generator 300 and manifold valve 302e. Fluid conduit 406 can be connected to manifold valve 302e and reaction vessel 310. Fluid conduit 408 can be connected to reaction vessel 310 and reaction vessel three-way solenoid valve 176, for example, through reactor pressure control port 118. Fluid conduit 410 can be connected to manifold valve 302a and solid phase cartridge 320. Fluid conduit 412 can be connected to solid phase cartridge 320 and manifold valve 304a. Fluid conduit 414 can be connected to manifold valve 302a and manifold valve 304d. Fluid conduit 416 can be connected to manifold valve 304b and buffer vessel 314. Fluid conduit 418 can be connected to manifold valve 304c and buffer vessel 314. Fluid conduit 420 can be connected to manifold valve 304a and final product vessel 316. Fluid conduit 422 can be connected to manifold valve 304e and waste vessel 312. Fluid conduit 424 can be connected to waste vessel 312 and waste vessel three-way solenoid valve 166, for example, through waste pressure control port 116. Pressure source 130 can also be connected to device 10, for example, through tubing connector 131. A sterile filter can be utilized to filter/sterilize the fluid as it flows into final product vessel 316. The sterile filter can be a vented Millipore™ filter (0.22 μm).

Buffer vessel 314 can be a 10 mL P15-041 lyophilized buffer vial. Buffer vessel 314 can contain 32 mg of sodium acetate tri-hydrate. In preparation for radiopharmaceutical generation, 6 mL of sterile saline can be added into buffer vessel 314. In addition, 1 mL of 0.2 N HCl solution can be added to buffer vessel 314.

Waste vessel 312 can be a 20 mL crimped vessel.

Solid phase cartridge 320 can be a NH2 light Sep-Pak. In preparation for radiopharmaceutical generation, solid phase cartridge 320 can be conditioned by passing at least 2 mL of ethanol USP and purging with approximately 10 mL of air. Solid phase cartridge 320 can then be rinsed with 5 mL of 0.05 N HCl solution and purged with approximately 10 mL of air.

Generator source reservoir 340 can contain at least 4 mL of 0.05 N HCl. First reagent reservoir 330 can be a 5 mL syringe. First reagent reservoir 330 can be filled with 3 mL of sterile water for injection and 1 mL of air. Second reagent reservoir 332 can be a 5 mL syringe. Second reagent reservoir 332 can be filled with 4 mL of sterile water for injection and 1 mL of air. Third reagent reservoir 334 can be a 5 mL syringe. Third reagent reservoir 334 can be filled with 2 mL of 0.1 N NaOH and 1 mL of air.

FIG. 14 illustrates an example method of automated operation of device 10.

At step 1401, a user supplies power to device 10, for example, but turning on power switch 108.

At step 1403, the user places selector switch 180 into a first position 181a representative of a test mode or a second position 181b representative of a radiopharmaceutical generation mode, i.e., run mode. If the selector switch is placed in first position 181a, the system proceeds to step 1405. If the selector switch is placed in second position 181b, the system proceeds to step 1411.

At step 1405, test indicator 182 can be activated, for example, by energizing a yellow LED.

Run indicator 184 can be maintained in an off state.

At step 1407, if the user has placed start switch 186 in an active state to provide an input to controller 60, the system can proceed to step 1409. If the start switch 186 is not in an active state, the system can proceed back to step 1403.

At step 1409, the system can execute a test sequence, as discussed in greater detail with respect to FIG. 15. After the test sequence, the system can return to step 1403.

At step 1411, run indicator 184 can be activated, for example, by energizing a green LED.

Test indicator 182 can be maintained in an off state.

At step 1413, if the user has placed start switch 186 in an active state to provide an input to controller 60, the system can proceed to step 1415. If the start switch 186 is not in an active state, the system can proceed back to step 1403.

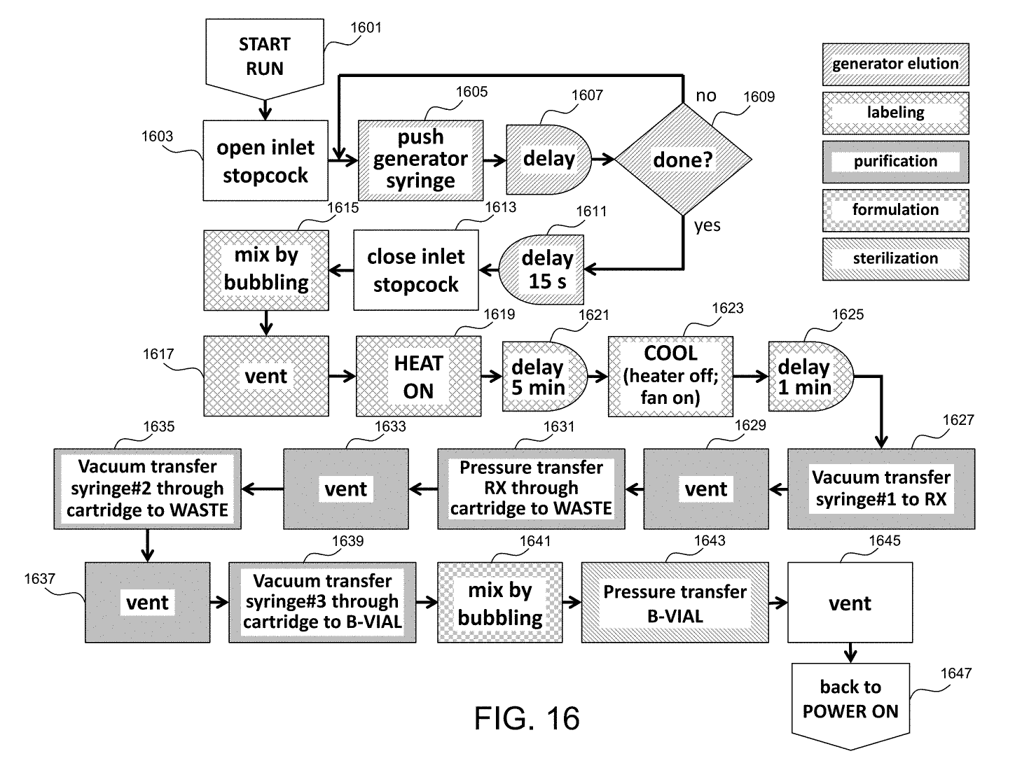

At step 1415, the system can execute a run sequence, as discussed in greater detail with respect to FIG. 16. After the run sequence, the system can return to step 1403.

An example method for an automated test procedure is shown in FIG. 15. Proper installation and of the hardware kit and other components can be confirmed before start of the manufacturing process through execution of a pre-programmed test procedure and observation of the machine's pressure gauge. The automated test procedure can take approximately four minutes to complete.

At step 1501, which corresponds to step 1409 in FIG. 14, device 10 can begin the test.

At step 1503, vacuum pump 120 can be activated and vacuum two-way solenoid valve 126 can be opened. Vacuum pump 120 can remain in an active state and vacuum two-way solenoid valve 126 can be opened for a specified amount of time at step 1505. At step 1507, vacuum pump 120 can be turned off and vacuum two-way solenoid valve 126 can be closed. At step 1509, a user can check the pressure reading on pressure gauge 160 to check the maximum vacuum of device 10 and to check for a leak in the system of device 10. For example, the maximum vacuum should be >24 mm Hg any change in system pressure should be less than approximately 1 mmHg or approximately 0.019 psig. At step 1511, the vacuum pressure in device 10 can be vented, for example, by opening vent two-way solenoid valve 156.

At step 1513, high-pressure from high-pressure regulator 132 can be activated and high-pressure two-way solenoid valve 136 can be opened. High-pressure can remain active and high-pressure two-way solenoid valve 136 can be opened for a specified amount of time at step 1515. At step 1517, high-pressure can be turned off and high-pressure two-way solenoid valve 136 can be closed. At step 1519, a user can check the pressure reading on pressure gauge 160 to check the high-pressure regulator set point and to check for a leak in the system of device 10. For example, any change in system pressure should be less than approximately 1.5 psig. At step 1521, the pressure in device 10 can be vented, for example, by opening vent two-way solenoid valve 156.

At step 1523, low-pressure from low-pressure regulator 142 can be activated and low-pressure two-way solenoid valve 146 can be opened. Low-pressure can remain active and low-pressure two-way solenoid valve 146 can be opened for a specified amount of time at step 1525. At step 1527, low-pressure can be turned off and low-pressure two-way solenoid valve 146 can be closed. A user can check the pressure reading on pressure gauge 160 to check the low-pressure regulator set point of the system of device 10. The pressure in device 10 can be vented.

At step 1529, manifold valve 304b can be opened to fluidly connect lower manifold 303 with fluid conduit 412, solid phase extraction cartridge 320 and fluid conduit 410. At step 1531, pressure can be provided to lower manifold 303 by activating the waste vessel three-way solenoid valve 166 and high-pressure two-way solenoid valve 136. At step 1533, pressure can be provided to lower manifold 303,fluid conduit 412, the solid phase extraction cartridge 302 and fluid conduit 410 for a specified amount of time. For example, lower manifold 303 can be pressurized to a pressure greater than approximately 10 psig. At step 1535, the high-pressure two-way solenoid valve 136 can be closed. At step 1537, a user can check the pressure reading on pressure gauge 160 to check for a leak in the system of device 10. At step 1539, the pressure in the lower cassette, fluid conduit 412, solid phase extraction cartridge 320 and fluid conduit 410 can be vented by deactivating the waste bottle three-way solenoid valve 166. Manifold valve 304b can be closed and any remaining pressure in device 10 can be vented by opening vent two-way solenoid valve 156 for a specified amount of time, for example 3 seconds.

At step 1541, pressure can be provided to upper manifold 301 and fluid conduit 414 by activating reactor vessel three-way solenoid valve 176 and opening high-pressure two-way solenoid valve 136. At step 1543, pressure can be provided to upper manifold 301 for a specified amount of time. For example, upper manifold 301 can be pressurized to a pressure greater than approximately 10 psig. At step 1545, the two-way solenoid valve. At step 1547, a user can check the pressure reading on pressure gauge 160 to check for a leak in the system of device 10. At step 1549, the pressure in the upper cassette and fluid conduit 414 can be vented by deactivating the three-way solenoid valve 176. Any remaining pressure in device 10 can be vented by opening vent two-way solenoid valve 156 for a specified amount of time., for example 3 seconds.

At step 1551, device 10 can return to a power on state, for example, step 1401 in FIG. 14.

Although the example method of FIG. 15 shows a particular order of steps, the exact order of the above steps could change (e.g., step 1519 could occur prior to step 1509), and the device 10 could receive additional input from the user before, after, and in between particular steps of the above example method.

An example method for an automated radiopharmaceutical labeling procedure is shown in FIG. 16.

In one aspect of the invention, device 10 can perform at least the following functions in an automated process: (1) introduce the radioactive labeling starting material; (2) mix the precursor labeling solution; (3) heat the labeling solution; (4) cool the labeling solution (5) purify the solution by solid phase extraction; (6) formulate the final product; and (7) perform sterile filtration of the final product. Although the system and methods are described herein for Ga-68, the system can be used with any suitable radionuclide and precursor molecule having compatible chemistry.

At step 1601, which corresponds to step 1415 in FIG. 14, device 10 can begin the radiopharmaceutical labeling procedure run sequence.

At step 1603, manifold valve 302e can be opened to fluidly connect fluid conduit 404 and fluid conduit 406 such that reaction vessel 310 is in fluid communication with generator 300.

At steps 1605-1609, device 10 can elute generator 300.

At step 1605, linear actuator 192 can be activated to move push plate 194 a specified distance and dispense a fluid into generator 300 from generator source reservoir 340 for elution of the radiopharmaceutical labeling material. At step 1607, device 10 can delay for a specified period of time as the fluid travels from generator source reservoir 340 into generator 300, through fluid conduit 404, manifold valve 302e, and fluid conduit 406 into reaction vessel 310. In this manner and in one aspect, linear actuator 192 can dispense fluid from generator source reservoir 340 at a rate of approximately 2 mL/min. At step 1609, device 10 can check whether the linear actuator has moved a specified distance corresponding to a specified amount of fluid having been dispensed from generator source reservoir 340 into generator 300 for elution of the radiopharmaceutical material. If an insufficient amount of fluid has been dispensed, device 10 can repeat steps 1605-1609. In one aspect, device 10 can dispense approximately 4 mL of 0.05 N HCl from generator source reservoir 340 to elute generator 300.

At step 1611, device 10 can delay a specified amount of time, for example, approximately 15 seconds.

At step 1613, manifold valve 302e can be closed such that fluid conduit 404 is not fluidly connected to fluid conduit 406.

At steps 1615-1625, device 10 can generate a radiopharmaceutical product.

At step 1615, the fluid in reaction vessel 310 can be mixed. Low-pressure two-way solenoid valve 146, waste vessel three-way solenoid valve 166 and manifold valve 304d can be opened such that low-pressure regulator 142 is in fluid communication with reaction vessel 310 via fluid conduits 424, 422, the lower cassette 303, 414 , the upper cassette 301 and 406. In this manner, low-pressure pressurized gas from pressure source 130 can mix the fluid within reaction vessel 310 by bubbling.

At step 1617, the system can be vented. Low-pressure two-way solenoid valve 146 can be closed can be closed, allowing the remaining pressure in fluid conduits 424,422, the lower cassette 303, 414, the upper cassette 301 and fluid conduit 406 to dissipate by bubbling through the solution in reaction vessel 310 until pressure has equilibrated with atmospheric pressure. Any remaining pressure in the manifold connected to pressure gauge 160 can be vented by opening the vent two-way solenoid valve 156 for a specified amount of time. For example, the vent two-way solenoid valve is opened for 3 seconds and then closed to relieve pressure.

At step 1619, temperature controlled reactor 102 can be activated to heat reaction vessel 310. In one aspect, temperature controlled reactor 102 can heat reaction vessel to approximately 70 degrees Celsius. During heating, upper cassette 301 can be pressurized to maintain the fluid within reaction vessel 310. In one aspect, low-pressure two-way solenoid valve 146, waste vessel three-way solenoid valve 166, manifold valve 304d and manifold valve 302a can be opened such that low-pressure regulator 142 is in fluid communication with the upper cassette 301 (up to manifold valve 302a) via fluid conduits 424, 422, the waste bottle 312, the lower cassette 303 and fluid conduit 414.

At step 1621, device can delay a specified amount of time, For example, temperature controlled reactor 102 can heat reaction vessel 310 for approximately 5 minutes at approximately 70 degrees Celsius.

At step 1623, temperature controlled reactor 102 can be deactivated and fan 104 can be activated to cool reaction vessel 310. During cooling the pressure to the upper cassette 301 can be relieved by venting through the fluid conduit 406 into reaction vessel 310. In one aspect, low-pressure two-way solenoid valve 146 and manifold valve 302a can be closed such that the remaining pressure in the waste bottle 312, lower cassette 303 and fluid conduits 424, 422, 414 and 406 vents through the reaction vessel 310. Finally, manifold valve 304d and waste three-way solenoid valve 166 can be closed to vent any pressure remaining waste bottle 312 and isolate the upper manifold 301 from the lower manifold 303. Any remaining pressure in the manifold connected to pressure gauge 160 can be vented by opening the vent two-way solenoid valve 156 for a specified amount of time. For example, the vent two-way solenoid valve is opened for 3 seconds and then closed to relieve pressure.

At step 1625, device 10 can delay a specified amount of time to allow for cooling of reaction vessel 310. For example, device 10 can delay for approximately 1 minute.

At steps 1627-1639, device 10 can purify the radiopharmaceutical product.

At step 1627, fluid from first reagent reservoir 330 can be transferred to reaction vessel 310. Vacuum pump 120 can be activated to generate a vacuum pressure. Vacuum two-way solenoid valve 126 can be opened and reaction vessel three-way solenoid valve 176 can be opened to fluidly connect a vacuum pump 120 and reaction vessel 310. Manifold valve 302d can be opened to fluidly connect first reagent reservoir 330 to fluid conduit 406 and reaction vessel 310. In this manner, the fluid within first reagent reservoir 330 can be dispensed into reaction vessel 310. In one aspect, the fluid within reactor vessel 310 can be diluted with approximately 3 mL of sterile water for injection from first reagent reservoir 330.

At step 1629, the system can be vented. Manifold valve 302d can be closed, vacuum pump 120 can be deactivated, vacuum two-way solenoid valve 126 can be closed, and/or reaction vessel three-way solenoid valve 176 can be closed. Remaining pressure in upper cassette 301, lower cassette 303, and/or the fluid conduits can be vented by opening reaction vessel three-way solenoid valve 176 to vent. Any remaining pressure in the manifold connected to pressure gauge 160 can be vented by opening the vent two-way solenoid valve 156 for a specified amount of time. For example, the vent two-way solenoid valve is opened for 3 seconds and then closed to relieve pressure.

At step 1631, the fluid within reaction vessel 310 can be passed through solid phase cartridge 320 and into waste vessel 312. Vacuum pump 120 can be activated to generate a vacuum pressure. Vacuum two-way solenoid valve 126 can be opened and waste vessel three-way solenoid valve 166 can be opened to fluidly connect vacuum pump 120 and waste vessel 310. Manifold valves 302a and 304a can be opened to fluidly connect reaction vessel 310, fluid conduit 406, upper cassette 301, fluid conduit 410, solid phase cartridge 320, fluid conduit 412, lower cassette 303, fluid conduit 422, and waste vessel 312. In this manner, vacuum pressure can draw the fluid from within reaction vessel 310 through solid phase cartridge 320 and into waste vessel 312. Solid phase cartridge 320 can retain the desired radiopharmaceutical material which is labeled with Ga-68, while the remaining fluid containing Ge-68 waste can flow into waste vessel 312.

At step 1633, the system can be vented. Vacuum pump 120 can be deactivated, vacuum two-way solenoid valve 126 can be closed, and/or waste vessel three-way solenoid valve 166 can be closed. Remaining pressure in upper cassette 301, lower cassette 303, and/or the fluid conduits can be vented by opening waste vessel three-way solenoid valve 166 to vent. Any remaining vacuum in the manifold connected to pressure gauge 160 can be vented by opening the vent two-way solenoid valve 156 for a specified amount of time. For example, the vent two-way solenoid valve is opened for 3 seconds and then closed to equalize pressure.

At step 1635, solid phase cartridge 320 can be rinsed with fluid from second reagent reservoir 332. Vacuum pump 120 can be activated to generate a vacuum pressure. Vacuum two-way solenoid valve 126 can be opened and waste vessel three-way solenoid valve 166 can be opened to fluidly connect a vacuum pump 120 and waste vessel 312. Manifold valves 302c, 302a and 304a can be opened to fluidly connect second reagent reservoir 332 to fluid conduit 410, solid phase cartridge 320, fluid conduit 412, lower cassette 303, fluid conduit 422, and waste vessel 312. In this manner, the fluid within second reagent reservoir 332 can be dispensed through solid phase cartridge 320 into waste vessel 312. In one aspect, solid phase cartridge 320 can be rinsed with approximately 4 mL of sterile water for injection from second reagent reservoir 332.

At step 1637, the system can be vented. Manifold valve 302c can be closed, vacuum pump 120 can be deactivated, vacuum two-way solenoid valve 126 can be closed, and/or waste vessel three-way solenoid valve 166 can be closed. Remaining pressure in upper cassette 301, lower cassette 303, and/or the fluid conduits can be vented by opening waste vessel three-way solenoid valve 166 to vent. Any remaining vacuum in the manifold connected to pressure gauge 160 can be vented by opening the vent two-way solenoid valve 156 for a specified amount of time. For example, the vent two-way solenoid valve is opened for 3 seconds and then closed to equalize pressure.

At step 1639, the desired radiopharmaceutical material can be eluted from solid phase cartridge 320 into buffer vessel 314. Vacuum pump 120 can be activated to generate a vacuum pressure. Vacuum two-way solenoid valve 126 can be opened and waste vessel three-way solenoid valve 166 can be opened to fluidly connect a vacuum pump 120 and waste vessel 312. Manifold valves 302b, 302a, 304a, 304b, and 304c can be opened to fluidly connect third reagent reservoir 334 to fluid conduit 410, solid phase cartridge 320, fluid conduit 412, lower cassette 303, fluid conduit 416, buffer vessel 314, fluid conduit 418, fluid conduit 422, and waste vessel 312. In this manner, the fluid within third reagent reservoir 334 can be dispensed through solid phase cartridge 320 to elute the desired radiopharmaceutical material from solid phase cartridge 320 into buffer vessel 314. In one aspect, solid phase cartridge 320 can be eluted with approximately 2 mL of 0.1 N NaOH from third reagent reservoir 334. Buffer vessel 314 can contain 6 mL of sterile saline, 1 mL of 0.2 N HCl, and 32 mg of sodium acetate trihydrate.

At step 1641, the radiopharmaceutical product within buffer vessel 314 can be mixed, for example, by bubbling with atmospheric gas. Manifold valve 302b can be closed, manifold valves 302a, 304a, 304b, and 304c can be opened, vacuum pump 120 can be deactivated, vacuum two-way solenoid valve 126 can be closed and waste vessel three-way solenoid valve 166 can be opened such that atmospheric gas is in fluid communication with the fluid conduit 312, reaction vessel 310, fluid conduit 406, upper cassette 301, fluid conduit 410, solid phase cartridge 320, fluid conduit 412, lower cassette 303. In this manner, atmospheric gas will mix the fluid within buffer vessel 314 by bubbling.

At step 1643, pressure can be provided to sterilize the radiopharmaceutical product by propelling the fluid within buffer vessel 314 into final product vessel 316 through a sterilizing filter. Low-pressure two-way solenoid valve 146 can be closed. High-pressure two-way solenoid valve 136 can be opened and manifold valves 304a, 304b, and 304c can be positioned such that high-pressure regulator 132 is in fluid communication with fluid conduit 424, waste vessel 312, fluid conduit 422, lower cassette 303, fluid conduit 418, buffer vessel 314, fluid conduit 416, fluid conduit 420, and final product vessel 316. The solution can be sterilized by passing through a 0.22 μm membrane filter as it flows into final product vessel 316.

At step 1645, the system can be vented. Manifold valve 304a can be closed such that final product vessel 316 is not in fluid communication with lower cassette 303. High-pressure two-way solenoid valve 136 can be closed, and/or waste vessel three-way solenoid valve 166 can be closed. Remaining pressure in upper cassette 301, lower cassette 303, and/or the fluid conduits can be vented by opening waste vessel three-way solenoid valve 166 to vent.

At step 1647, device 10 can return to a power on state, which corresponds to step 1401 in FIG. 14.

The total time for device 10 to execute steps 1601-1647 can be approximately 16 minutes.

After waiting an appropriate length of time to allow for the decay of the machine components, device 10 can be prepared for generator by removing the single-use hardware kit and installing a new kit.

It is to be appreciated that the Detailed Description section, and not the Summary and

Abstract sections, is intended to be used to interpret the claims. The Summary and Abstract sections may set forth one or more but not all exemplary embodiments of the present invention(s) as contemplated by the inventor(s), and thus, are not intended to limit the present invention(s) and the appended claims in any way.

The present invention(s) have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention(s) that others can, by applying knowledge within the skill of the art, readily modify and/or adapt for various applications such specific embodiments, without undue experimentation, without departing from the general concept of the present invention(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention(s) should not be limited by any of the above-described exemplary embodiments, but should be defined only in accordance with the following claims and their equivalents.

### EXAMPLES

The following Examples are provided to illustrate, not to limit, aspects of the present invention.

### Example 1

### P15-041 Radiolabeling by Automated Machine

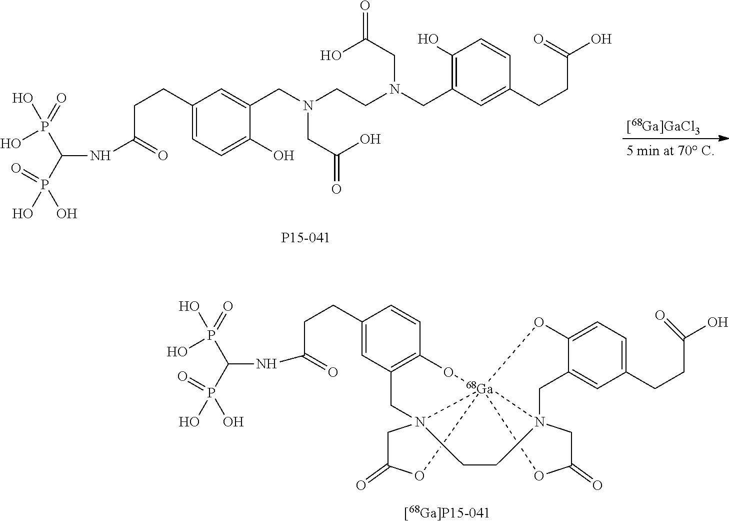

Preparation of [68Ga]P15-041 begins with the elution of the 68Ge/68Ga generator with 4 mL of 0.05 N HCl into the lyophilized drug substance precursor “kit”. The solution is heated to approximately 70° C. for 5 minutes using a temperature regulated heat source. After heating for 5 minutes the solution is allowed to cool for 1 min by removing power to the heater and flowing room temperature air with a fan. The cooled solution is diluted with water and then passed through an activated NH2 Sep Pak® light cartridge. The drug substance is retained on the cartridge while some 68Ge impurity passes through into a waste container. The cartridge is rinsed with water to complete the purification process. The drug substance is eluted from the cartridge using 2 mL of 0.1 N NaOH and is immediately mixed with 1 mL of 0.2 N HCl and 6 mL of saline and ˜32 mg sodium acetate trihydrate to adjust the pH and tonicity of the solution. Most of the remaining 68Ge impurity is retained on the NH2 cartridge. The solution is then sterilized by passing through a 0.22 μm membrane filter.

### Example 2

### P15-071 Radiolabeling by Automated Machine

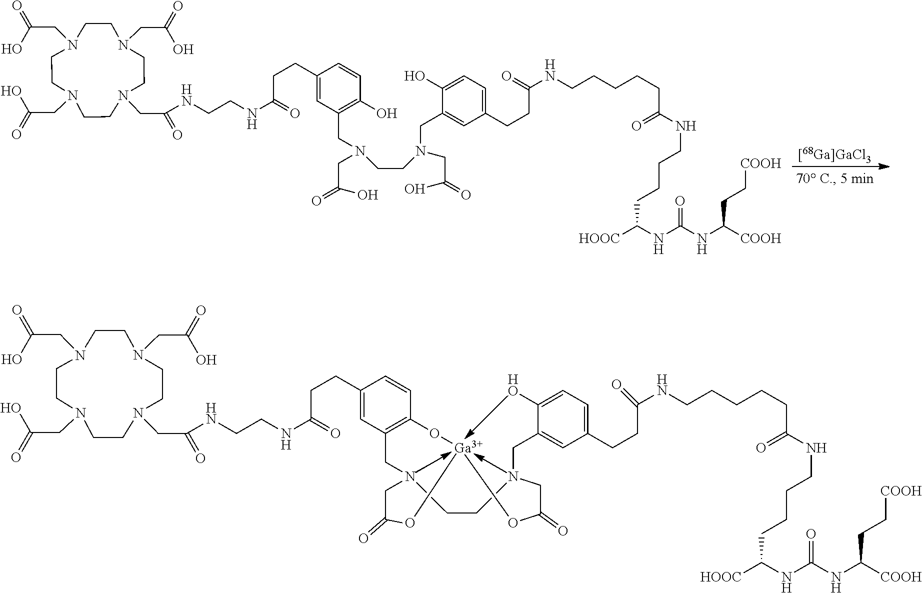

Preparation of [68Ga]P15-071 begins with the elution of the 68Ge/68Ga generator with 4 mL of 0.05 N HCl into the lyophilized drug substance precursor “kit”. The solution is heated to approximately 70° C. for 5 minutes using a temperature regulated heat source. After heating for 5 minutes the solution is allowed to cool for 1 min by removing power to the heater and flowing room temperature air with a fan. The cooled solution is diluted with water and then passed through an activated Chromafix C8 (M) cartridge. The drug substance is retained on the cartridge while some 68Ge impurity passes through into a waste container. The cartridge is rinsed with water to complete the purification process. The drug substance is eluted from the cartridge using 2 mL of 30% ethanol in saline and is immediately mixed with 7 mL of sterile saline to adjust the volume and tonicity of the solution. Most of the remaining 68Ge impurity is retained on the C8 cartridge. The solution (˜9 mL) is then sterilized by passing through a 0.22 μm membrane filter.

### Example 3

### P16-093 Radiolabeling by Automated Machine

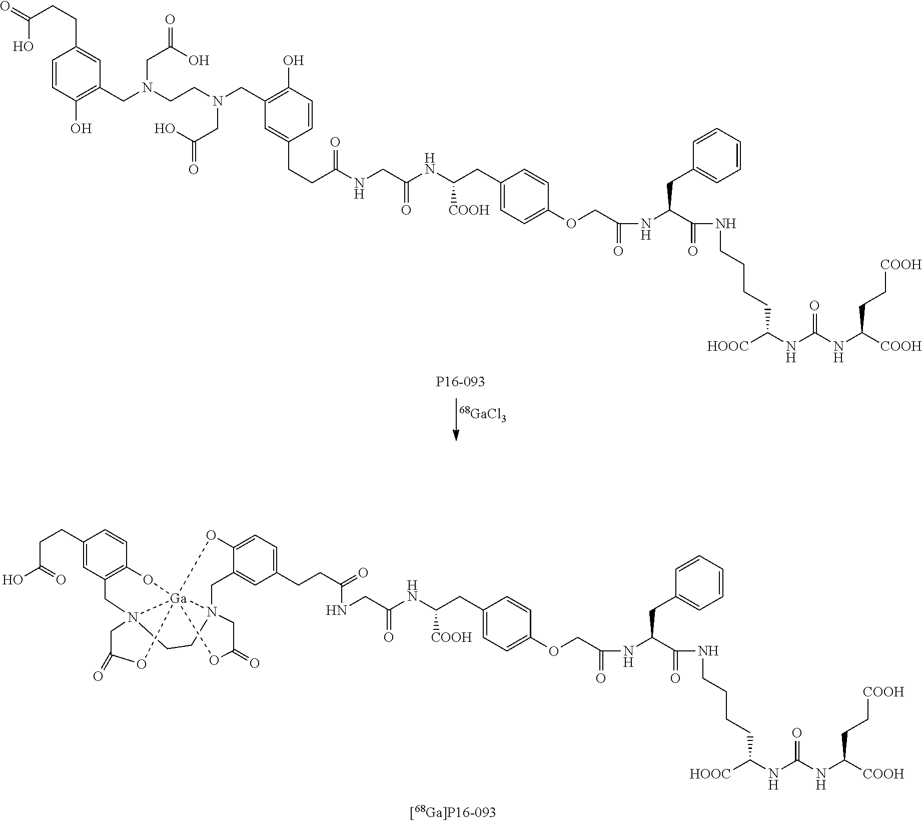

Preparation of [68Ga]P16-093 begins with the elution of the 68Ge/68Ga generator with 4 mL of 0.05 N HCl into the lyophilized drug substance precursor “kit”. The solution is heated to approximately 70° C. for 5 minutes using a temperature regulated heat source. After heating for 5 minutes the solution is allowed to cool for 1 min by removing power to the heater and flowing room temperature air with a fan. The cooled solution is diluted with water and then passed through an activated Chromafix C8 (M) cartridge. The drug substance is retained on the cartridge while some 68Ge impurity passes through into a waste container. The cartridge is rinsed with water to complete the purification process. The drug substance is eluted from the cartridge using 2 mL of 40% ethanol in saline and is immediately mixed with 7 mL of sterile saline to adjust the volume and tonicity of the solution. Most of the remaining 68Ge impurity is retained on the C8 cartridge. The solution (˜9 mL) is then sterilized by passing through a 0.22 μm membrane filter.

What is claimed is:

1. An automated method to synthesize a radiopharmaceutical, the automated method comprising:
passing a fluid through a radioactive labeling generator to introduce the radioactive labeling starting material into a precursor labeling solution;
mixing the precursor labeling solution;
heating and cooling the labeling solution;
purifying the solution by solid phase extraction;
formulating a final radiopharmaceutical product; and
performing sterile filtration of the final radiopharmaceutical product.

2. The automated method of claim 1, wherein:
the radioactive labeling generator is a 68Ge/68Ga generator,
the labeling solution is heated for approximately 70 degrees Celsius for approximately 5 minutes, and
the final radiopharmaceutical product is formulated by eluting an extracted drug substance using 2 mL of 0.1 N NaOH and mixing with 1 mL of 0.2 N HCl, 6 mL of saline, and approximately 32 mg sodium acetate trihydrate.

3. An automated apparatus for producing a radiopharmaceutical, the apparatus comprising:
an outer housing
a temperature controlled reactor; and
a valve manifold comprising a plurality of manifold valves, the valve manifold being detachably attached to the outer housing and being fluidly connected to:
a radiopharmaceutical reactor, the radiopharmaceutical reactor being fluidly connected to a reactor reservoir, the reactor reservoir containing a generator fluid to elute a material from the radiopharmaceutical reactor,
a reaction vessel positioned within the temperature controlled reactor, the temperature controlled reactor being configured to heat the reaction vessel,
a solid phase extraction device to extract the material from the generator fluid,
a reagent reservoir containing a reagent fluid to elute the material from the solid phase extraction device, and
a final radiopharmaceutical vessel to receive the reagent fluid and the material.

4. The apparatus of claim 3, further comprising:
a high-pressure regulator fluidly connected to a pressure source and a high-pressure valve;
a low-pressure regulator fluidly connected to the pressure source and a low-pressure valve;
a vacuum pump; and
a pressure measurement device,
wherein the valve manifold is fluidly connected to:
the high-pressure valve,
the low-pressure valve,
the vacuum pump, and
the pressure measurement device.

5. The apparatus of claim 4, further comprising a controller to control operation of the high-pressure valve, the low-pressure valve, the vacuum pump, the temperature controlled reactor, and the plurality of manifold valves.

6. The apparatus of claim 5, wherein during operation of the automated apparatus, the controller does not receive input from a computing device.

7. The apparatus of claim 5, wherein operation of the automated apparatus is commenced by an input signal to the controller.

8. The apparatus of claim 7, wherein the input signal is generated by a mechanical switch.

9. The apparatus of claim 3, wherein each of the plurality of manifold valves is a three way valve.

10. The apparatus of claim 3, wherein the valve manifold is a stopcock manifold and each of the plurality of manifold valves is a multi-way stopcock valve.

11. The apparatus of claim 3, wherein one of the plurality of manifold valves includes a valve handle, wherein the valve manifold is detachably attached to the outer housing via a valve adapter that is detachably attached to the valve handle.

12. The apparatus of claim 11, further comprising a motor attached to the valve adapter to rotate the valve adapter,
wherein rotation of the valve adapter changes a position of the valve handle and an operating position of the valve.

13. The apparatus of claims 12, further comprising:
a generator fluid pump to move the generator fluid through the radiopharmaceutical reactor; and
a controller to control operation of the motor and the generator fluid pump,
wherein during operation of the automated apparatus, the controller does not receive input from a computing device.

14. A valve adapter to detachably attach a valve handle to a motor, the valve adapter having a length extending along a first direction and a width extending along a second direction, the second direction being perpendicular to the first direction, the valve adapter comprising:
a central body having a valve end and a motor end, the valve end and the motor end being spaced along the first direction;
a first cantilever member attached to the central body at the valve end and a first cantilever member first end, the first cantilever member extending from the central body in the second direction to a first cantilever member second end;
a second cantilever member attached to the central body at the valve end and a second cantilever member first end, the second cantilever member extending from the central body in the second direction to a second cantilever member second end;
a protrusion attached to the first cantilever member, the protrusion being positioned between the first cantilever member and the second cantilever member.

15. The valve adapter of claim 14, wherein the protrusion is configured to engage the valve handle to detachably attach the valve handle to the valve adapter.

16. The valve adapter of claim 14, further comprising a second protrusion attached to the second cantilever member, the second protrusion being positioned between the first cantilever member and the second cantilever member,
wherein the second protrusion is configured to engage the valve handle to detachably attach the valve handle to the valve adapter.

17. The valve adapter of claim 14, further comprising a channel to receive a second valve handle, the channel extending along a third direction, the third direction being perpendicular to the first direction and the second direction.

18. The valve adapter of claim 17, further comprising a dual tapered guide surface positioned adjacent the channel to direct the second valve handle into the channel, the dual tapered guide surface being tapered in the first direction and in the third direction.

19. The valve adapter of claim 17, further comprising a second channel to receive a third valve handle, the second channel extending along the third direction.

20. The valve adapter of claim 14, further comprising a spline inset in the motor end of the central body, the spline being configured to engage a gear of the motor.
